(19) United States
(12) Patent Application Publication
(10) Pub. No.: US 2020/0197876 A1
(43) Pub. Date: Jun. 25, 2020

(54) METHOD FOR DETERMINING CHANNEL SIZES, MEMBRANE THICKNESSES, INLET PRESSURES, OUTLET SPLITTER LOCATIONS AND/OR CO2 PRESSURES AND OTHER VARIABLES FOR A DIFFUSIOPHORETIC WATER FILTRATION DEVICE, AND RELATED DEVICE AND TEST CENTER

(71) Applicant: Split Rock Filter Systems LLC, New Paltz, NY (US)

(72) Inventor: William C. Gehris, New Paltz, NY (US)

(21) Appl. No.: 16/724,822

(22) Filed: Dec. 23, 2019

Related U.S. Application Data
(60) Provisional application No. 62/783,493, filed on Dec. 21, 2018.
(60) Provisional application No. 62/783,848, filed on Dec. 21, 2018.
(60) Provisional application No. 62/784,578, filed on Dec. 24, 2018.
(60) Provisional application No. 62/785,403, filed on Dec. 27, 2018.
(60) Provisional application No. 62/788,106, filed on Jan. 3, 2019.

(51) Int. Cl. B01D 65/10; C02F 1/44; C02F 1/00; B01D 61/02; G01N 33/18

(52) U.S. Cl. CPC B01D 65/10; C02F 1/441; G01N 33/18; B01D 61/025; C02F 1/008

(57) ABSTRACT

A test center for collecting data related to a diffusiophoretic water filtration module. A method for improving the water quality includes determining at least one undesired colloidal particle and operating a test diffusiophoretic filtration device for effectiveness in removing the undesired colloidal particle. A method for designing a diffusiophoretic water filter also includes analyzing data on colloidal particulates removed from water having passed through a diffusiophoretic water filter having known physical characteristics and known operating conditions; and determining an importance of at least one of the colloidal particulates with respect to at least one of the physical conditions and the known operating conditions.

This claims the benefit of U.S. Provisional Patent Application Nos. 62/783,493, filed on Dec. 21, 2018, 62/783,848, filed Dec. 21, 2018, 62/784,578, filed on Dec. 24, 2018, 62/785,403, filed on Dec. 27, 2018 and 62/788,106, filed on Jan. 3, 2019. All of the above listed applications are hereby incorporated in their entirety herein.

### BACKGROUND

WO 2018/048735 discloses a device operative in separating particles in a flowing suspension of the particles in a liquid which device comprises: a first, pressurized cavity or plenum adapted to contain a gas, separated by a first gas permeable wall from a second cavity or plenum which contains a charged particle containing liquid which also contains an ion species formed by the dissolution of the gas within the liquid, which is in turn separated by a second permeable wall from the ambient atmosphere or an optional, third, relatively reduced pressure cavity or plenum which may contain a gas or a vacuum; wherein: the permeable walls operate to permit for the transfer of a gas from the first cavity through the second cavity and through the second permeable wall to the atmosphere or a third cavity and, the pressure present in atmosphere or the third cavity is lesser than that of the first cavity, thus forming an ion concentration differential within the liquid and between the permeable walls.

The related article “Membraneless water filtration using CO2” by Shin et al. (Nature Communications 8:15181), 2 May 2017, describes a continuous flow particle filtration device in which a colloidal suspension flows through a straight channel in a gas permeable material made of polydimethylsiloxane (PDMS). A CO2 (carbon dioxide) gas channel passes parallel to the wall and dissolves into the flow stream. An air channel on the other side of the wall prevents saturation of CO2 in the suspension and the resulting gradient of CO2 causes particles to concentrate on sides of the channel, with negatively charged particles moving toward the air channel and positively charged particles toward the CO2 channel. The water away from the sides of the channel can be collected as filtered water.

The article “Diffusiophoresis at the macroscale” by Mauger et al. (arXiv: 1512.05005v4), 6 July 2016, discloses that solute concentration gradients caused by salts such as LiCl impact colloidal transport at lengthscales ranging roughly from the centimeter down to the smallest scales resolved by the article. Colloids of a diameter of 200 nm were examined.

The article “Origins of concentration gradients for diffusiophoresis” by Velegol et al, (10.1039/c6sm00052e), 13 May 2016, describes diffusiophoresis possibly occurring in georeservoir extractions, physiological systems, drying operations, laboratory and industrial separations, crystallization operations, membrane processes, and many other situations, often without being recognized.

PCT Publication WO 2015/077674 discloses a process that places a microparticle including a salt in proximity to a membrane such that the microparticle creates a gradient generated spontaneous electric field or a gradient generated spontaneous chemiphoretic field in the solvent proximal to the membrane. This gradient actively draws charged particles, via diffusiophoresis, away from the membrane thereby removing charged particulate matter away from the membrane or preventing its deposition.

### SUMMARY OF THE INVENTION

The present applicant has developed modular diffusiophoretic water filters that are easily scalable to thousands of units. At least one sheet in the device has a surface structured with longitudinal channels having a general cross sectional shape that can be defined generally by a height and width. International Patent Application PCT/US18/61146, filed on Nov. 14, 2018 published as WO 2019/099586, U.S. Pat. No. 10,155,182 issued on Dec. 18, 2018 and U.S. Pat. No. 10,463,994, issued on Nov. 5, 2019 and PCT Application No. PCT/US19/65976, filed on Dec. 12, 2019 are all hereby incorporated by reference herein, in their entirety.

One of the greatest challenges of diffusiophoretic water filtration technology is that every municipal or ground water or other water source to be filtered in a real world application will differ due to both, a varying temperature and due to different colloidal particles as well as other factors such as dissolved ion content such as salt.

It is difficult to select or optimize a proper channel length, height and width, and if gas is being used to drive the diffusiophoretic action, CO2 pressure and the CO2 membrane thickness, as well as an air membrane thickness for a closed channel needs to be selected. For ion exchange driven water filters, ion-exchange membrane characteristics such as thickness and swellability can alter diffusiophoretic action. The proper channel length for all devices also can depend on inlet pressures, outlet splitter location, temperature, atmospheric pressure of the device location, and possible other variables.

The approach to date has been to model the diffusiophoretic action of various particles and to try to design channels based on a desired diffusiophoretic action, for example using mathematical modeling.

The present invention provides a data-based analytical approach to determining channel sizes, CO2 pressure, inlet pressure membrane thickness, and/or other variables for a diffusiophoretic water filter.

The present invention provides a data center running with at least one but preferably a plurality, for example hundreds or thousands, of small scale diffusiophoretic water filters, for example each one module producing 25 ml/hour of filtered water.

Each can collect data on filtering of different water sources, for example some with municipal waters with known or tested chemical compositions, so distilled water with added known concentrations of at least one colloid, sometimes with salt added, sometimes not. The conditions such as temperature, channel sizes, CO2 pressure, membrane thickness etc. can vary for each module and test point.

The output of each can be analyzed, and thus data on the effectiveness of diffusiophoretic action for each test condition obtained for different water chemistries.

A 200 day operating year of a test center running 1000 modules each with 5 tests a day can produce a million separate test results.

As the data collected becomes large, it can be analyzed using rather simple artificial intelligence and/or statistical data analysis. Multivariate analysis such as multiple regression analysis or factor analysis can be used.

A municipality then seeking to reduce E. coli content in its water source then can present its water chemistry, say with a certain salt, E. coli and other chemical content. Various water temperatures and other environmental data can also be provided.

Optimal channel sizes and operating conditions based on the existing database then be determined or suggested. These sizes and operating conditions can then be used on a test module to determine accuracy and the test module simply used as a basis for a commercial device.

The general channel dimensions preferably vary between 20 cm and 1.2 meters in length, 20 micrometers to 1 mm in thickness and 20 micrometers to 5 cm in width. The CO2 pressure preferably varies between 1.1 and 2 atm, and the colloid input pressure between 1 and 100 mbar. The PDMS membrane thickness for CO2 and air distance with the colloid channel preferably varies between 10 and 80 micrometers.

The operating variables to be selected, such as the channel size and inlet pressure, thus can number less than 10. The number of colloids and other water chemistry variables for which data is determined can be for example 10 to 100.

The present invention thus provides the following:

a test center for collecting data related to a diffusiophoretic water filtration module;

a database collecting data related to a diffusiophoretic water filtration module for use in designing a commercial water filter;

a method for analyzing a database to determine structures or operating conditions of a diffusiophoretic water filtration module;

a method for testing a water source and based on data analysis, determining suggested characteristics for a test module to be used as a basis for larger diffusiophoretic water filter;

a method comprising gathering data from a plurality of diffusiophoretic water filtration test modules operating under different test conditions, the method for improving the water quality for municipal water supply comprising: determining at least one undesired colloidal particle in a municipal water source; operating a test diffusiophoretic filtration device for effectiveness in removing the undesired colloidal particle; providing information about, or supplying or requiring the supply of, diffusiophoretic filtration devices with similar diffusiophoretic action as the test diffusiophoretic filtration device to a plurality of drinking water recipients of the municipal source;

a method for improving the water quality for municipal water supply comprising: determining at least one undesired colloidal particle in a municipal water source; operating a test diffusiophoretic filtration device with a test diffusiophoretic channel having a known channel size for effectiveness in removing the undesired colloidal particle; providing information about, or supplying or requiring the supply of, diffusiophoretic filtration devices with at least one channel being a same size as the test diffusiophoretic channel to a plurality of drinking water recipients of the municipal source; and

a diffusiophoretic water filter sheet wider than a test sheet with a same channel size.

The present invention can have particular applicability with respect to RIO water filters, which often have specifically known clogants. The present invention thus also provides a water filter comprising a modular diffusiophoretic water filter (DWF) and an RIO water filter, the diffusiophoretic water filter being upstream of the R/O water filter.

The present invention also provides a method for providing a DWF upstream of an R/O water filter, the DWF having a channel structure designed to eliminate at least one colloidal particle known to be a clogant for the R/O water filter.

The channel structure of the DWF can be selected by identifying at least one colloidal particle, and preferably several of the actual or most likely colloidal clogants, and running tests on a test DWF with one or more smaller test modules to determine the most efficient channel structure and/or operating characteristics of the DWF, such as operating temperature, membrane thickness, CO2 pressure, inlet pressure and outlet splitter location.

The present invention also provides a method for providing a DWF upstream of an R/O water filter, the DWF being as a function of a database to reduce expected or actual clogants in the R/O water filter.

The RIO device thus can operate more efficiently and remove ionic components such as K+, Na+ and other particles that are not capable of being removed by the DWF.

### BRIEF DESCRIPTION OF THE DRAWINGS

The present invention is described with respect to embodiments described in the following schematic drawing in which:

FIG. 1 shows a test center with a plurality of diffusiophoretic water filter (DWF) test modules, and a database for collecting information from the water filters;

FIGS. 2 and 3 show test membranes with different channel widths, lengths and heights;

FIG. 4 shows a differing replaceable wall structure in a test module to provide seals for differing test membranes;

FIG. 5 shows a DWF test module schematically; and

FIG. 6 shows a modular DWF used in a R/O system.

### DETAILED DESCRIPTION

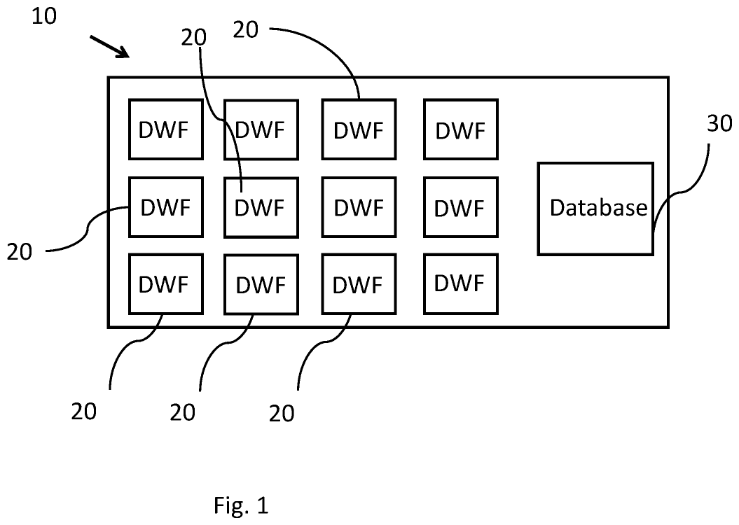

FIG. 1 shows a test center 10 with a plurality of diffusiophoretic water filter (DWF) test modules 20, and a database 30 for collecting information from the water filters.

Each test module 20 can be a DWF with for example differing characteristics, and hundreds or thousands, of small scale diffusiophoretic water filters, for example each one module producing 25 ml/hour of filtered water, can be provided.

Each module 20 can collect data on filtering of different water sources, for example some with municipal waters with known or tested chemical compositions, so distilled water with added known concentrations of at least one colloid, sometimes with salt added, sometimes not. The conditions including as input pressure, flow velocity, dwell time, temperature, channel sizes including width thickness and length, CO2 pressure, membrane thickness, and splitter ratio can vary for each module 20 and each test.

The output of each can be analyzed for each water source, and thus data on the effectiveness of diffusiophoretic action for each test condition obtained for different water chemistries.

A 200 day operating year of a test center running 1000 modules each with 5 tests a day can produce a million separate test results.

As the data collected becomes large, it can be analyzed using rather simple artificial intelligence and/or statistical data analysis. Multivariate analysis such as multiple regression analysis or factor analysis can be used.

The effect of test conditions such as input pressure, flow velocity, dwell time, temperature, channel sizes including width thickness and length, CO2 pressure, membrane thickness, and splitter ratio can be analyzed with respect to for example each and every colloidal particulate in the water source, for example E. coli, plastic nanoparticle or a PFOA or other chemical attached to a nanoparticle introduces upstream in the DWF module 20, as described for example in copending U.S. patent application Ser. No. 16/262,633, filed on Jan. 30, 2019 hereby incorporated by reference herein.

Thus for example the importance of for example dwell time can be identified and related to the removal of E. coli, as compared to its importance with respect to plastic nanoparticles, and the importance of dwell time with respect to CO2 pressure can be identified with respect to removal of nanoparticles.

This knowledge, based on a large data set of preferably thousands of tests, advantageously allows for development and optimization of DWFs, especially modular and scalable DWFs.

The test modules 20 need not be adjustable with respect to their physical characteristics, but in a preferred embodiment they are.

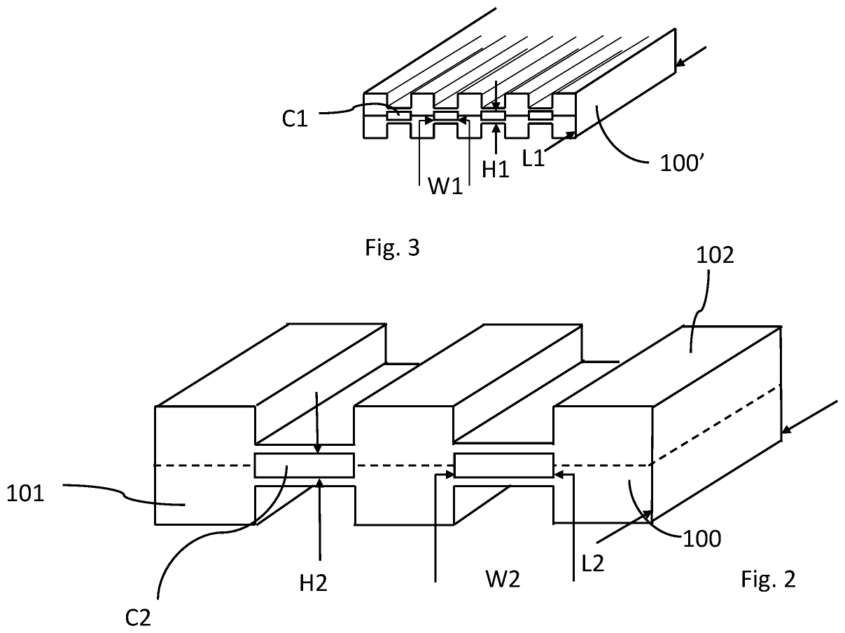

FIGS. 2 and 3 show an embodiment of such adjustable test modules 20, having test membranes 100, 100′ with different channel widths, lengths and heights. As shown in FIG. 2, membrane 100 may be a two part membrane 101, 102 that when put together form channels C2, each with a height H2, width W2 and length L2. FIG. 3 shows a second membrane 100′ with channels C1, each with a height H1, width W1 and length L1.

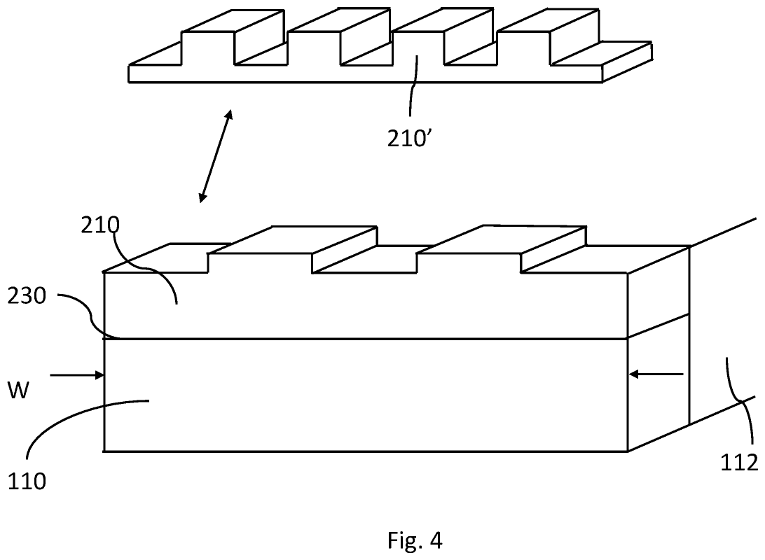

FIG. 4 shows a replaceable wall structure for a test module 20 to provide seals for differing test membranes. Seal structure 210 can be replaced with a seal structure 210′, and still fit in a same side wall 112. A seal 230 can be provided between seal structures 210, 210′ and a front wall 110.

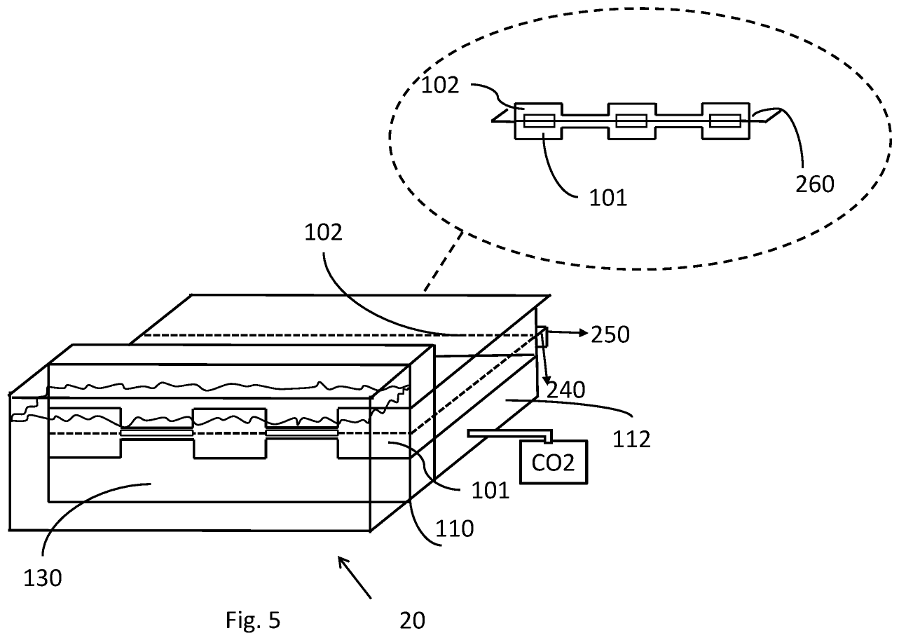

FIG. 5 shows a DWF test module schematically, attached to a reservoir or inlet manifold 130, which can set an inlet pressure for the channels C2, C1, and showing a CO2 source where the pressure can be set, and shown the outlet splitter 260 creating a filtered (clean) water stream 240 and a waste water stream 250. The splitter ratio can vary for example by providing different thickness partial channels in membranes 101 and 102.

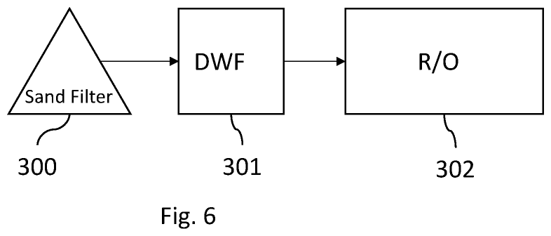

FIG. 6 shows a modular DWF used in a RIO system, with a sand filter 300, a DWF 301 designed as discussed above and reverse osmosis water filter 302.

What is claimed is:

1. A test center for collecting data related to a diffusiophoretic water filtration module.

2. A method for improving the water quality comprising:
determining at least one undesired colloidal particle;
operating a test diffusiophoretic filtration device for effectiveness in removing the undesired colloidal particle.

3. A method for designing a diffusiophoretic water filter comprising:
analyzing data on colloidal particulates removed from water having passed through a diffusiophoretic water filter having known physical characteristics and known operating conditions;
determining an importance of at least one of the colloidal particulates with respect to at least one of the physical conditions and the known operating conditions.
